(19) United States
(12) Patent Application Publication
(10) Pub. No.: US 2021/0378536 A1
(43) Pub. Date: Dec. 9, 2021

(54) PULSE DETECTION APPARATUS AND PULSE DETECTION METHOD

(71) Applicant: Panasonic Corporation, Osaka (JP)

(72) Inventors: Takuma IKEDA, Osaka (JP); Yuki TANAKA, Tokyo (JP); Hiroyuki TANI, Hyogo (JP)

(21) Appl. No.: 17/335,417

(22) Filed: Jun. 1, 2021

(30) Foreign Application Priority Data
Jun. 3, 2020 (JP) 2020-096962

(51) Int. Cl. A61B 5/024; A61B 5/00

(52) U.S. Cl. CPC A61B 5/02444; A61B 5/742; A61B 5/7405

(57) ABSTRACT

Provided is a pulse detection apparatus. The apparatus includes: a pulse wave detector that detects a pulse wave, and outputs a pulse wave signal indicating the pulse wave that has been detected; a threshold value setting circuit that sets a threshold value that changes with time in accordance with an amplitude of the pulse wave signal; and a comparator that compares the pulse wave signal with the threshold value, and outputs, as information indicating a pulse, the pulse wave signal exceeding the threshold value.

### TECHNICAL FIELD

The present disclosure relates to a pulse detection apparatus and a pulse detection method.

### BACKGROUND ART

In recent years, as the aging society advances, demand for watching over people has been growing, and pulse detection using a wearable terminal has been attracting attention. In the pulse detection using a wearable terminal, it is also important to ensure convenience, and a small and lightweight battery is preferably mounted on the wearable terminal.

Patent Literature (hereinafter, referred to as “PTL”) 1 discloses a technique that uses an analog circuit to realize pulse detection while suppressing an increase in power consumption. A pulse wave detection apparatus of PTL 1 includes a photoelectric pulse wave detector, an amplitude determiner, a notifier, and the like. A pulse wave signal that has been detected by the photoelectric pulse wave detector is inputted as an analog signal, which vibrates around its base-line voltage, into the amplitude determiner. The amplitude determiner into which the analog signal is inputted shifts the base-line voltage of the analog signal to a ground level. Further, the amplitude determiner outputs the analog signal, whose base-line voltage has been shifted to the ground level, to the notifier. The notifier generates a sound synchronized with a beat at a timing at which the amplitude of the pulse wave signal is equal to or larger than a threshold value set in advance.

### CITATION LIST

### Patent Literature

PTL 1

Japanese Patent Application Laid-Open No. 2009-201801

### SUMMARY OF INVENTION

### Technical Problem

In the prior art of PTL 1, however, a pulse wave signal is collated with a threshold value with a constant amplitude, and therefore variations in pulse wave signals due to external factors, such as individual differences in the intensity of pulse waves and installation positions of sensors, are not taken into consideration. Accordingly, there is room for improvement in terms of realizing stable pulse detection.

One non-limiting and exemplary embodiment facilitates providing a pulse detection apparatus and a pulse detection method that are capable of realizing stable pulse detection.

### Solution to Problem

A pulse detection apparatus according to an embodiment of the present disclosure includes: a pulse wave detector that detects a pulse wave, and outputs a pulse wave signal indicating the pulse wave that has been detected; a threshold value setting circuit that sets a threshold value that changes with time in accordance with an amplitude of the pulse wave signal; and a comparator that compares the pulse wave signal with the threshold value, and outputs, as information indicating a pulse, the pulse wave signal exceeding the threshold value.

A pulse detection method according to the embodiment of the present disclosure includes: detecting a pulse wave, and outputting a pulse wave signal indicating the pulse wave that has been detected; setting a threshold value that changes with time in accordance with an amplitude of the pulse wave signal; and comparing the pulse wave signal with the threshold value, and outputting, as information indicating a pulse, the pulse wave signal exceeding the threshold value.

### Advantageous Effects of Invention

According to the embodiment of the present disclosure, it is possible to construct a pulse detection apparatus and a pulse detection method that are capable of realizing stable pulse detection.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF DRAWINGS

FIG. 1 illustrates a configuration example of pulse detection apparatus 101 according to an embodiment of the present disclosure; 1

FIG. 2 illustrates a configuration example of pulse detection circuit 103; 2

FIG. 3 illustrates waveforms of pulse wave signal 201 (V_in) and signal 203 (α×V_in) 3;

FIG. 4 illustrates examples of signal 203 (α×V_in), threshold value 206 (V_SH), and threshold value 208 (β×V_SH) 4; and

FIG. 5 illustrates pulse wave signal 201 (V_in), and threshold value 1 with a constant amplitude.

### DESCRIPTION OF EMBODIMENTS

Hereinafter, a preferred embodiment of the present disclosure will be described in detail with reference to the accompanying drawings. Note that, in the present specification and drawings, components having substantially the same function are denoted by the same reference numerals, and redundant description will be omitted.

### Embodiment

<Configuration Example of Pulse Detection Apparatus 101>

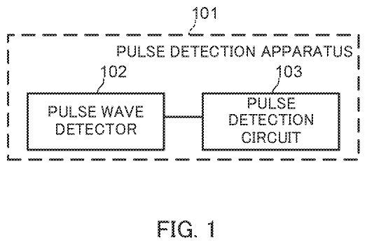

FIG. 1 illustrates a configuration example of pulse detection apparatus 101 according to an embodiment of the present disclosure. Pulse detection apparatus 101 includes pulse wave detector 102 and pulse detection circuit 103.

Pulse wave detector 102 is a photoelectric sensor, a piezoelectric sensor or the like that is used in proximity to a human body. Pulse wave detector 102 is attached to, for example, an arm of a person, and detects a pulse wave of the person.

Pulse detection circuit 103 is an analog circuit that detects a pulse wave based on a signal indicating the pulse wave that has been detected by pulse wave detector 102.

<Pulse Detection Circuit 103>

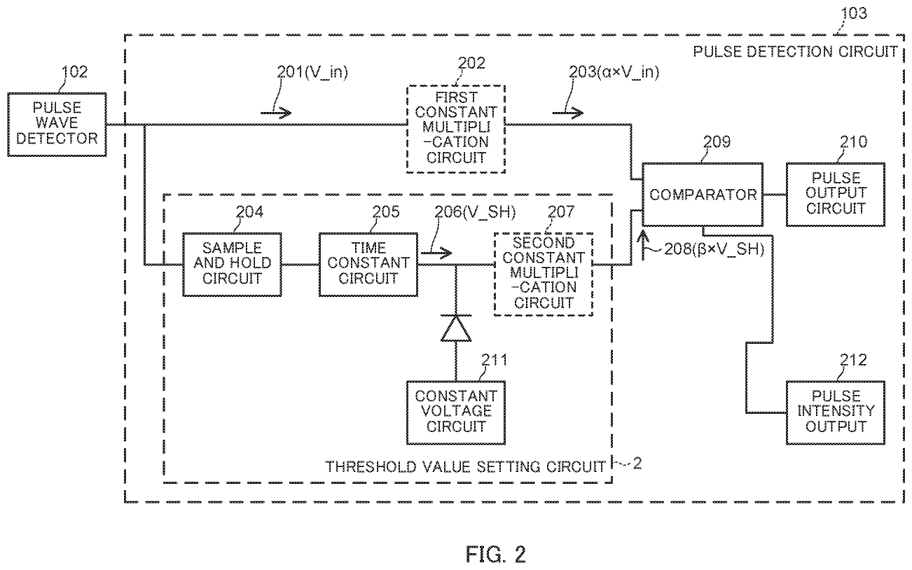

Next, a configuration example of pulse detection circuit 103 with reference to FIG. 2. FIG. 2 is the configuration example of pulse detection circuit 103 5.

Pulse detection circuit 103 includes first constant multiplication circuit 202, sample and hold circuit 204, time constant circuit 205, second constant multiplication circuit 207, comparator 209, pulse output circuit 210, constant voltage circuit 211, and pulse intensity output 212. Sample and hold circuit 204, time constant circuit 205, second constant multiplication circuit 207, and constant voltage circuit 211 form threshold value setting circuit 2. Threshold value setting circuit 2 is an analog circuit that sets threshold values (threshold value 206 (V_SH) and threshold value 208 (β×V_SH)) that change with time in accordance with an amplitude of pulse wave signal 201 (V_in).

<First Constant Multiplication Circuit 202>

First constant multiplication circuit 202 inputs pulse wave signal 201 (V_in) that is a signal indicating a pulse wave that has been detected by pulse wave detector 102. First constant multiplication circuit 202 multiplies pulse wave signal 201 (V_in), which is inputted into first constant multiplication circuit 202, by coefficient α larger than 1 (for example, 1.1 to 3.0, or the like) to generate signal 203 (α×V_in), which is amplified, and outputs signal 203 (α×V_in) to comparator 209.

<Sample and Hold Circuit 204>

Sample and hold circuit 204 inputs pulse wave signal 201 (V_in), generates a signal in which a peak value of pulse wave signal 201 (V_in) is held, and outputs the signal to time constant circuit 205.

<Time Constant Circuit 205>

Time constant circuit 205 maintains a signal, which is inputted into time constant circuit 205, for a specific time constant, thereby outputting the inputted signal as information indicating threshold value 206 (V_SH) to second constant multiplication circuit 207.

<Second Constant Multiplication Circuit 207>

Second constant multiplication circuit 207 multiplies threshold value 206 (V_SH), which is inputted into second constant multiplication circuit 207, by coefficient β smaller than 1 (for example, 0.1 to 0.9, or the like) to generate threshold value 208 (β×V_SH), which is attenuated, and outputs threshold value 208 (β×V_SH) to comparator 209.

<Comparator 209>

Comparator 209 inputs signal 203 (α×V_in) and threshold value 208 (β×V_SH), and outputs, as information indicating a pulse, signal 203 (Δ×V_in) exceeding threshold value 208 (β×V_SH) to pulse output circuit 210.

<Pulse Output Circuit 210>

Based on information indicating a pulse, which is inputted into pulse output circuit 210, pulse output circuit 210 generates a sound in accordance with the pulse from a speaker, and visualizes the pulse to cause the visualized pulse to be displayed on an indicator, for example.

<Constant Voltage Circuit 211>

Constant voltage circuit 211 is a circuit that sets a lower limit value of a voltage of threshold value 206 (V_SH) maintained by time constant circuit 205. An output terminal of constant voltage circuit 211 is connected to an output terminal of time constant circuit 205 via a diode. By application of an output voltage of constant voltage circuit 211 to the output terminal of time constant circuit 205, threshold value 206 (V_SH) is maintained at a predetermined value even in a case where the output (threshold value 206 (V_SH)) of time constant circuit 205 is small.

Thus, for example, even in a case where an amplitude of pulse wave signal 201 (V_in) is small due to installation of pulse wave detector 102 in a site of a body, where it is difficult to detect a pulse wave, it is possible to restrain threshold value 206 (V_SH) from being buried in a noise or the like and to perform stable pulse detection.

<Pulse Intensity Output 212>

Pulse intensity output 212 has a function of outputting the intensity of a pulse that has been detected by comparator 209. Pulse intensity output 212 may be, for example, a sound generating means, such as a buzzer and a speaker, which generates a sound in accordance with a pulse intensity, or may be a display or the like which displays an image in accordance with a pulse intensity.

<Pulse Detection Operation>

Next, a pulse detection operation of pulse detection circuit 103 will be described.

Pulse wave signal 201 (V_in) that has been detected by pulse wave detector 102 is inputted into first constant multiplication circuit 202 and sample and hold circuit 204, respectively.

Pulse wave signal 201 (V_in) inputted into first constant multiplication circuit 202 is multiplied by coefficient α larger than 1 to generate signal 203 (α×V_in) in which pulse wave signal 201 (V_in) is amplified. Signal 203 (α×V_in) is inputted into comparator 209. Specific examples of waveforms of signal 203 (Δ×V_in) will be described later.

A peak value of pulse wave signal 201 (V_in) inputted into sample and hold circuit 204 is held by sample and hold circuit 204, and is further maintained for a predetermined time by time constant circuit 205. Thus, threshold value 206 (V_SH) is generated and is inputted into second constant multiplication circuit 207. Threshold value 206 (V_SH) inputted into second constant multiplication circuit 207 is multiplied by coefficient β smaller than 1 to generate threshold value 208 (β×V_SH) in which threshold value 206 (V_SH) is attenuated.

In comparator 209 into which signal 203 (α×V_in) and threshold value 208 (β×V_SH) are inputted, signal 203 (α×V_in) exceeding threshold value 208 (β×V_SH) is detected as a pulse.

For detection of a pulse, it is necessary to satisfy a relationship of signal 203 (α×V_in) >threshold value 208 (β×V_SH), for which it is necessary to satisfy a relationship of coefficient α>coefficient β.

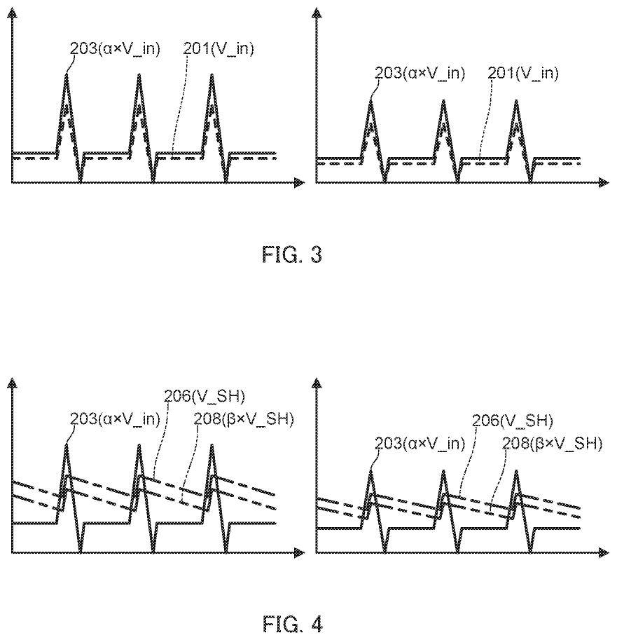

A waveform of pulse wave signal 201 (V_in) before multiplication by coefficient α and a waveform of signal 203 (α×V_in) after multiplication by coefficient α will be described with reference to FIG. 3.

FIG. 3 illustrates waveforms of pulse wave signal 201 (V_in) and signal 203 (α×V_in) 6. A diagram on the left side of FIG. 3 illustrates signal 203 (α×V_in) generated when pulse wave signal 201 (V_in) having a large amplitude is detected. A diagram on the right side of FIG. 3 illustrates signal 203 (α×V_in) generated when pulse wave signal 201 (V_in) having a small amplitude is detected.

As illustrated in FIG. 3, in both cases where the amplitude of pulse wave signal 201 (V_in) is large and where the amplitude of pulse wave signal 201 (V_in) is small, the waveform of signal 203 (α×V_in) is similar to the waveform of pulse wave signal 201 (V_in).

Next, each waveform of signal 203 (α×V_in), threshold value 206 (V_SH), and threshold value 208 (β×V_SH) will be described with reference to FIG. 4.

FIG. 4 illustrates examples of signal 203 (α×V_in), threshold value 206 (V_SH), and threshold value 208 (β×V_SH) 7.

On the left side of FIG. 4, each waveform of signal 203 (α×V_in), threshold value 206 (V_SH), and threshold value 208 (β×V_SH) generated when pulse wave signal 201 (V_in) having a large amplitude is detected is illustrated.

On the right side of FIG. 4, each waveform of signal 203 (α×V_in), threshold value 206 (V_SH), and threshold value 208 (β×V_SH) generated when pulse wave signal 201 (V_in) having a small amplitude is detected is illustrated.

As illustrated in FIG. 4, the sizes of threshold value 206 (V_SH) and threshold value 208 (β×V_SH) change with time in accordance with the size of a pulse, that is, the amplitude of signal 203 (α×V_in).

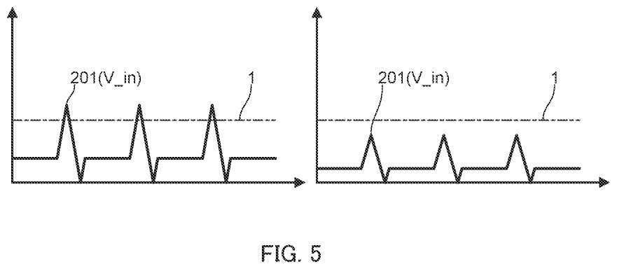

Conventional pulse detection apparatuses, on the other hand, utilize a threshold value with a constant amplitude. This threshold value will be described with reference to FIG. 5. FIG. 5 illustrates pulse wave signal 201 (V_in), and threshold value 1 with a constant amplitude.

On the left side of FIG. 5, pulse wave signal 201 (V_in) having a large amplitude, and threshold value 1 with a constant amplitude are illustrated. As seen therein, in the case of pulse wave signal 201 having a large amplitude, the peak value of pulse wave signal 201 exceeds threshold value 1, and it is therefore possible to perform stable pulse detection.

On the right side of FIG. 5, pulse wave signal 201 (V_in) having a small amplitude, and threshold value 1 with a constant amplitude are illustrated. As seen therein, in the case of pulse wave signal 201 having a large amplitude, the peak value of pulse wave signal 201 does not exceed threshold value 1, and it is therefore difficult to perform stable pulse detection.

Pulse detection apparatus 101 according to the present embodiment is configured such that threshold value 208 (β×V_SH) which changes with time in accordance with each amplitude of pulse wave signal 201 (V_in) and signal 203 (α×V_in) is set. Accordingly, even in a case where the amplitude of pulse wave signal 201 (V_in) is small, it is possible to perform stable pulse detection by utilizing threshold value 208 that changes with time.

Further, the prior art utilizes an A/D converter with relatively large power consumption, whereas pulse detection apparatus 101 according to the present embodiment includes comparator 209, and an analog circuit such as a sample and hold circuit 204, and therefore makes it possible to perform stable pulse detection while suppressing an increase in power consumption.

Further, given the capability to suppress an increase in power consumption, it is possible to combine pulse detection apparatus 101 according to the present embodiment with a small and lightweight battery. Accordingly, application of pulse detection apparatus 101 according to the present embodiment to a wearable apparatus using a piezoelectric sensor or the like makes it possible to realize a small and lightweight wearable apparatus capable of performing pulse detection.

For example, it is understood that the following aspects also fall within the technical scope of the present disclosure.

(1) A pulse detection apparatus includes: a pulse wave detector that detects a pulse wave, and outputs a pulse wave signal indicating the pulse wave that has been detected; a threshold value setting circuit that sets a threshold value that changes with time in accordance with an amplitude of the pulse wave signal; and a comparator that compares the pulse wave signal with the threshold value, and outputs, as information indicating a pulse, the pulse wave signal exceeding the threshold value.

(2) The pulse detection apparatus further includes a first constant multiplication circuit that constant-multiplies the pulse wave signal by a value larger than 1, where the pulse wave signal indicates the pulse wave. The threshold value setting circuit includes: a sample and hold circuit that holds a peak value of the pulse wave signal that has been detected by the pulse wave detector; and a time constant circuit that maintains the peak value for a predetermined time, and outputs the peak value as the threshold value, where the peak value has been held by the sample and hold circuit. The comparator compares the pulse wave signal with the threshold value, where the pulse wave signal has been constant-multiplied by the first constant multiplication circuit, and the threshold value has been outputted from the sample and hold circuit.

(3) The threshold value setting circuit includes: a sample and hold circuit that holds a peak value of the pulse wave signal that has been detected by the pulse wave detector; a time constant circuit that maintains the peak value for a predetermined time, and outputs the peak value as the threshold value, where the peak value has been held by the sample and hold circuit; and a second constant multiplication circuit that constant-multiplies the threshold value by a value smaller than 1, where the threshold value has been outputted from the time constant circuit. The comparator compares the pulse wave signal with the threshold value, where the pulse wave signal has been outputted from the pulse wave detector, and the threshold value has been constant-multiplied by the second constant multiplication circuit.

(4) The pulse detection apparatus further includes a first constant multiplication circuit that constant-multiplies the pulse wave signal by a value larger than 1, where the pulse wave signal indicates the pulse wave. The threshold value setting circuit includes: a sample and hold circuit that holds a peak value of the pulse wave signal that has been detected by the pulse wave detector; a time constant circuit that maintains the peak value for a predetermined time, and outputs the peak value as the threshold value, where the peak value has been held by the sample and hold circuit; and a second constant multiplication circuit that constant-multiplies the threshold value by a value smaller than 1, where the threshold value has been outputted from the time constant circuit. The comparator compares the pulse wave signal with the threshold value, where the pulse wave signal has been constant-multiplied by the first constant multiplication circuit, and the threshold value has been constant-multiplied by the second constant multiplication circuit.

(5) The pulse detection apparatus further includes a constant voltage circuit that sets a lower limit value of the threshold value outputted from the time constant circuit.

(6) A pulse detection method includes: detecting a pulse wave, and outputting a pulse wave signal indicating the pulse wave that has been detected; setting a threshold value that changes with time in accordance with an amplitude of the pulse wave signal; and comparing the pulse wave signal with the threshold value, and outputting, as information indicating a pulse, the pulse wave signal exceeding the threshold value.

(7) The pulse detection method includes: constant-multiplying the pulse wave signal by a value larger than 1; holding a peak value of the pulse wave signal; maintaining the peak value for a predetermined time, and outputting the peak value as the threshold value, where the peak value has been held; and comparing the pulse wave signal with the threshold value, where the pulse wave signal has been constant-multiplied, and the threshold value has been maintained for the predetermined time.

(8) The pulse detection method includes: holding a peak value of the pulse wave signal; maintaining the peak value for a predetermined time, and outputting the peak value as the threshold value, where the peak value has been held; constant-multiplying the threshold value by a value smaller than 1, where the threshold value has been maintained for the predetermined time; and comparing the pulse wave signal with the threshold value that has been constant-multiplied.

(9) The pulse detection method includes: constant-multiplying the pulse wave signal by a value larger than 1; holding a peak value of the pulse wave signal; maintaining the peak value for a predetermined time, and outputting the peak value as the threshold value, where the peak value has been held; constant-multiplying the threshold value by a value smaller than 1, where the threshold value has been maintained for the predetermined time; and comparing the pulse wave signal with the threshold value, where the pulse wave signal has been constant-multiplied, and the threshold value has been constant-multiplied.

While various embodiments have been described herein above, it is to be appreciated that various changes in form and detail may be made without departing from the sprit and scope of the invention(s) presently or hereafter claimed.

This application is entitled and claims the benefit of Japanese Patent Application No. 2020-096962, filed on Jun. 3, 2020, the disclosure of which including the specification, drawings and abstract is incorporated herein by reference in its entirety.

### INDUSTRIAL APPLICABILITY

The embodiment of the present disclosure is suitable for a pulse detection apparatus.

### REFERENCE SIGNS LIST

1: Threshold value

2: Threshold value setting circuit

101: Pulse detection apparatus

102: Pulse wave detector

103: Pulse detection circuit

201: Pulse wave signal

202: First constant multiplication circuit

203: Signal

204: Sample and hold circuit

205: Time constant circuit

206: Threshold value

207: Second constant multiplication circuit

208: Threshold value

209: Comparator

210: Pulse output circuit

211: Constant voltage circuit

212: Pulse intensity output

1. A pulse detection apparatus, comprising:
a pulse wave detector that detects a pulse wave, and outputs a pulse wave signal indicating the pulse wave that has been detected;
a threshold value setting circuit that sets a threshold value that changes with time in accordance with an amplitude of the pulse wave signal; and
a comparator that compares the pulse wave signal with the threshold value, and outputs, as information indicating a pulse, the pulse wave signal exceeding the threshold value.

2. The pulse detection apparatus according to claim 1, further comprising a first constant multiplication circuit that constant-multiplies the pulse wave signal by a value larger than 1, the pulse wave signal indicating the pulse wave, wherein:
the threshold value setting circuit includes:
a sample and hold circuit that holds a peak value of the pulse wave signal that has been detected by the pulse wave detector; and
a time constant circuit that maintains the peak value for a predetermined time, and outputs the peak value as the threshold value, the peak value having been held by the sample and hold circuit, and
the comparator compares the pulse wave signal with the threshold value, the pulse wave signal having been constant-multiplied by the first constant multiplication circuit, the threshold having been outputted from the sample and hold circuit.

3. The pulse detection apparatus according to claim 1, wherein:
the threshold value setting circuit includes:
a sample and hold circuit that holds a peak value of the pulse wave signal that has been detected by the pulse wave detector;
a time constant circuit that maintains the peak value for a predetermined time, and outputs the peak value as the threshold value, the peak value having been held by the sample and hold circuit; and
a second constant multiplication circuit that constant-multiplies the threshold value by a value smaller than 1, the threshold value having been outputted from the time constant circuit, and
the comparator compares the pulse wave signal with the threshold value, the pulse wave signal having been outputted from the pulse wave detector, the threshold value having been constant-multiplied by the second constant multiplication circuit.

4. The pulse detection apparatus according to claim 1, further comprising a first constant multiplication circuit that constant-multiplies the pulse wave signal by a value larger than 1, the pulse wave signal indicating the pulse wave, wherein:
the threshold value setting circuit includes:
a sample and hold circuit that holds a peak value of the pulse wave signal that has been detected by the pulse wave detector;
a time constant circuit that maintains the peak value for a predetermined time, and outputs the peak value as the threshold value, the peak value having been held by the sample and hold circuit; and
a second constant multiplication circuit that constant-multiplies the threshold value by a value smaller than 1, the threshold value having been outputted from the time constant circuit, and
the comparator compares the pulse wave signal with the threshold value, the pulse wave signal having been constant-multiplied by the first constant multiplication circuit, the threshold value having been constant-multiplied by the second constant multiplication circuit.

5. The pulse detection apparatus according to any one of claim 2, further comprising a constant voltage circuit that sets a lower limit value of the threshold value outputted from the time constant circuit.

6. A pulse detection method, comprising:
detecting a pulse wave, and outputting a pulse wave signal indicating the pulse wave that has been detected;
setting a threshold value that changes with time in accordance with an amplitude of the pulse wave signal; and
comparing the pulse wave signal with the threshold value, and outputting, as information indicating a pulse, the pulse wave signal exceeding the threshold value.

7. The pulse detection method according to claim 6, comprising:
constant-multiplying the pulse wave signal by a value larger than 1;
holding a peak value of the pulse wave signal;
maintaining the peak value for a predetermined time, and outputting the peak value as the threshold value, the peak value having been held; and
comparing the pulse wave signal with the threshold value, the pulse wave signal having been constant-multiplied, the threshold value having been maintained for the predetermined time.

8. The pulse detection method according to claim 6, comprising:
holding a peak value of the pulse wave signal;
maintaining the peak value for a predetermined time, and outputting the peak value as the threshold value, the peak value having been held;
constant-multiplying the threshold value by a value smaller than 1, the threshold value having been maintained for the predetermined time; and
comparing the pulse wave signal with the threshold value that has been constant-multiplied.

9. The pulse detection method according to claim 6, comprising:
constant-multiplying the pulse wave signal by a value larger than 1;
holding a peak value of the pulse wave signal;
maintaining the peak value for a predetermined time, and outputting the peak value as the threshold value, the peak value having been held;
constant-multiplying the threshold value by a value smaller than 1, the threshold value having been maintained for the predetermined time; and
comparing the pulse wave signal with the threshold value, the pulse wave signal having been constant-multiplied, the threshold value having been constant-multiplied.
